(19) United States
(12) Patent Application Publication
(10) Pub. No.: US 2024/0079145 A1
(43) Pub. Date: Mar. 7, 2024

(54) SYSTEMS AND METHODS FOR DERIVING HEALTH INDICATORS FROM USER-GENERATED CONTENT

(71) Applicant: MY LUA LLC, Albany, NY (US)

(72) Inventors: Michael CONWARD, Miami, FL (US); J'Vanay SANTOS-FABIAN, Los Angeles, CA (US); U-Leea SANTOS-FABIAN, Los Angeles, CA (US)

(73) Assignee: MY LUA LLC, Albany, NY (US)

(21) Appl. No.: 18/261,194

(22) PCT Filed: Jan. 15, 2022
(86) PCT No.: PCT/US2022/012645

§ 371 Date: Jul. 12, 2023

Related U.S. Application Data
(60) Provisional application No. 63/138,204, filed on Jan. 15, 2021.

(51) Int. Cl. G16H 50/30; G06F 40/40; G16H 10/60; G16H 20/00; G16H 40/67; G16H 50/20; G16H 50/70

(52) U.S. Cl. CPC G16H 50/30; G06F 40/40; G16H 10/60; G16H 20/00; G16H 40/67; G16H 50/20; G16H 50/70

(57) ABSTRACT

The present disclosure relates to systems and methods for generating priority lists and/or predictions or identifications of root causes of acute or chronic conditions. In one exemplary embodiment, a method comprises aggregating data corresponding to a plurality of individuals, the data comprising, for each individual, user-generated content and/or biometric data; generating, from a machine learning model that utilizes the aggregated user-generated content and/or biometric data as input, one or more of a priority list for the plurality of individuals, or, for each individual, a prediction, diagnosis, or identification of one or more root causes of one or more acute or chronic conditions of the individual.

### CROSS-REFERENCE TO RELATED APPLICATION(S)

This application is a United States National Stage Application of International Application No. PCT/US2022/012645, filed on Jan. 15, 2022 and published on Jul. 21, 2022 as WO 2022/155555 A1, which claims the benefit of priority of U.S. Provisional Patent Application No. 63/138,204, filed on Jan. 15, 2021, each of which is hereby incorporated by reference herein in its entirety for any purpose whatsoever.

### TECHNICAL FIELD

The present disclosure relates to systems for health monitoring and evaluation, and, more specifically, to systems and methods that utilize machine learning models that utilize patient biometric data.

### BACKGROUND

Postpartum depression is a severe public health problem and has a reported occurrence rate of 10-20 percent among new mothers in the United States. Not only does this condition negatively impact the physical and mental health of the mother, but it can also be detrimental to the emotional and cognitive development of the child, sometimes leading to suicide and infanticide. Many at-risk mothers have limited access to healthcare providers or simply do not seek help for their mental health symptoms due to discomfort with conventional interventions, such as pharmaceutical products. Poor sleep conditions and social determinants of health (SDoH) are also well-known factors associated with maternal morbidity, such as preterm birth, gestational hypertension, preeclampsia, and gestational diabetes. Additionally, racial disparities exist among these adverse obstetric outcomes, and although SDoH have been proposed as main contributors, reasons remain uncertain.

Non-pharmacological, complementary, and alternative medicine (CAM) has been increasingly sought out among women, including at-risk populations in the United States that include women of color, ethnic minorities, and low-income women. Research studies involving CAM, such as yoga, meditation, and journaling, have demonstrated significant symptom reduction, yet more research is needed in order to prove the effectiveness of CAM techniques before they can be recommended to substitute or complement other evidence-based treatments.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be exemplary only.

FIG. 1 illustrates an exemplary system architecture in accordance with at least one embodiment of the present disclosure.

FIG. 2 is a flow diagram illustrating a training process for training machine learning models in accordance with at least one embodiment of the present disclosure.

FIG. 3 illustrates an exemplary dashboard presented for display by a user interface of a personnel device in accordance with at least one embodiment of the present disclosure.

FIG. 4 illustrates an exemplary dashboard illustrating patient information and patient relationships in accordance with at least one embodiment of the present disclosure.

FIG. 5 illustrates an exemplary dashboard for displaying a patient's biometric data other data derived therefrom in accordance with at least one embodiment of the present disclosure.

FIG. 6 is a flow diagram illustrating a modeling process for predicting risk and performing risk assessment in accordance with at least one embodiment of the present disclosure.

FIG. 7 is a flow diagram illustrating a method of deriving health indicators from user-generated content in accordance with at least one embodiment.

FIG. 8 is a flow diagram illustrating a method of generating priority lists and/or predictions of root causes of acute or chronic conditions in accordance with at least one embodiment.

FIG. 9 is a block diagram illustrating an exemplary computer system in accordance with at least one embodiment.

### DETAILED DESCRIPTION

Disclosed herein are systems and methods for deriving health indicators based at least partially from user-generated content and/or biometric data. Health indicators can include, but are not limited to, predictions, diagnosis, or identifications of root causes of acute or chronic health (e.g., physical or mental health) conditions, and risk scores and categories (e.g., relating to a probability that a patient may have or develop a health condition). Certain embodiments relate to the use of risk scores and categories for generating priority lists of patients, which are provided to clinicians, health plan care managers, or other healthcare stakeholders to facilitate treating or mitigating root causes of the physical or mental health symptoms, such as those related to pregnancy. Moreover, symptom escalation alerts may be generated and sent to relevant personnel.

Certain embodiments may utilize content generated by a patient, such as textual data (e.g., journal entries written by the patient), audio data (e.g., the patient's voice, from which the content and tone can be analyzed), survey data (e.g., standard health surveys completed by the patient), or image or video data (e.g., video of the patient's face or body, images of handwriting, etc., from which physical movement or facial expressions can be analyzed). Other data may be utilized in connection with user-generated content, including, but not limited to, patient electronic medical record (EMR) data and social determinants of health (SDoH) data.

In some embodiments, natural language processing (NLP) modeling may be used to identify words or phrases descriptive of various physical or mental health symptoms, as well as perform mood or sentiment analysis. The resulting data may be used in combination with or in lieu of responses to standard health surveys, and associated with biometric data to identify root causes underlying the patient's symptoms. In some embodiments, the associations may be processed to provide, for example, CAM recommendations to the patient to mitigate or treat the symptoms and/or underlying conditions.

While current approaches generally analyze physical and mental health of a patient separately, the embodiments of the current disclosure can advantageously analyze both physical and mental health concurrently. Moreover, embodiments of the present disclosure seek to facilitate the study of the effects of digitized versions of CAM (e.g., yoga, meditation, and journaling) on pregnant or postpartum women.

The amount of incoming health data in high volume can overwhelm health care professionals, leading to treatment delays or clinical errors. The application of machine learning can provide highly accurate and timely decision making capabilities for supporting health care needs. In some embodiments, a machine learning platform may process health data using one or more approaches including, but not limited to, neural networks, decision tree learning, deep learning, etc. For applications pertaining to the health of pregnant or postpartum women in particular, data collected and derived from online journaling exercises can provide clearer insight into the mother's experience, as opposed to standard surveys with ratings on predetermined questions. Moreover, machine learning models that can associate such data with biometric data (e.g., collected by one or more wearable devices) can be used to directly identify, detect, and predict physical and mental health conditions specific to postpartum women.

Certain embodiments also relate to HIPAA- and HITRUST-compliant patient mobile applications to allow for individuals (e.g., patients such as individuals in a pregnancy-related period) to regularly log their mood, complete health risk assessment tests (e.g., Edinburgh Postnatal Depression Scale (EPDS), Patient Health Questionair-9 (PHQ-9), questions or Generalized Anxiety Test Questionnaire (GAD-7) questions), track their symptoms, and capture and monitor relevant biometric data using wearable devices or contactless sensors. Data entered directly by users into the mobile application (referred to herein as “user-generated content”), biometric data, and EMR data may be continuously captured and utilized as inputs to one or more machine learning models. In certain embodiments, outputs of the one or more machine learning models include, but are not limited to, patient risk scores, which assess risks related to physical health and mental health, that can be provided to personnel, such as clinicians, for visualization. In some embodiments, the risk scores may be associated with risks of patients developing postpartum depression.

Certain embodiments also provide a dashboard (which may be in the form of a mobile application) to clinicians, health plan care managers, or other healthcare stakeholders that can be used to visualize and track patient appointments, visualize and monitor patient biometric data, provide insights and alerts regarding patient risk, and allow for direct messaging with patients.

Advantages of the embodiments of the present disclosure include, but are not limited to: (1) reduced depression/anxiety in patients, including pregnant or postpartum women; (2) data collection in underserved or at-risk populations for need identification; (3) ongoing screening and preventative care capabilities; (4) tools for implementing self-care and self-assessment; (5) patient-specific product and clinician matching; (6) comprehensive identification and prediction of underlying physical and mental health conditions; (7) mitigation of instances of missed or delayed diagnosis; (8) streamlined and accelerated EMR data integration; (8) performing data transformations and mappings that are compliant with the HL7® FHIR® standard; (10) automation of clinical workflows to provide end-to-end data liquidity; and (11) compliance with the SMART-on-FHIR platform to facilitate and simplify last-mile integration with health system EMR data.

Although embodiments of the present disclosure are discussed in terms of health monitoring and management, the embodiments may also be generally applied to other applications including drug or alcohol abuse treatment, grief counseling, etc.

As used herein, the term “pregnancy-related period” refers to periods of time that may include the actual period of pregnancy, the period starting from child planning (including attempts at conception) up until conception, and the postpartum period.

Also as used herein, the term “postpartum period” can refer to a period beginning at childbirth and ending at a particular time. The postpartum period may extend, for example, from 1 month, 2 months, 3 months, etc., up to 12 or 36 months from childbirth.

Also as used herein, the term “pregnancy-related symptom” refers to any symptom of known or unknown physical or mental health conditions that occur for a patient during the pregnancy-related period.

Also as used herein, the term “user-generated content” refers to any information generated by an individual by means of a user device that includes, but is not limited to, textual data, survey data (e.g., survey responses), audio data, image or video data, or any other data voluntarily generated by the individual from which information about the individual can be extracted.

Also as used herein, the term “biometric data” refers to any data descriptive of or derived from a measurable physiological quantity associated with an individual. Non-limiting examples of biometric data include heart rate data, body temperature data, body composition data (e.g., body mass index, percent body fat, etc.), hemoglobin level data, cholesterol data, sleep data, blood pressure data, respiratory rate data, blood glucose level data, triglyceride data, movement data, electrodermal activity data, electrocardiogram data, electroencephalograph data, or other parameters. Biometric data may be generated from wearable devices as well as from contactless sensors. In some embodiments, biometric data may also be obtained in the form of a user input into a user device or personnel device rather than as data obtained directly from a biometric measurement device (for example, temperature data may be obtained as a result of an individual measuring their own temperature with a thermometer and then reporting the measurement via their personal device).

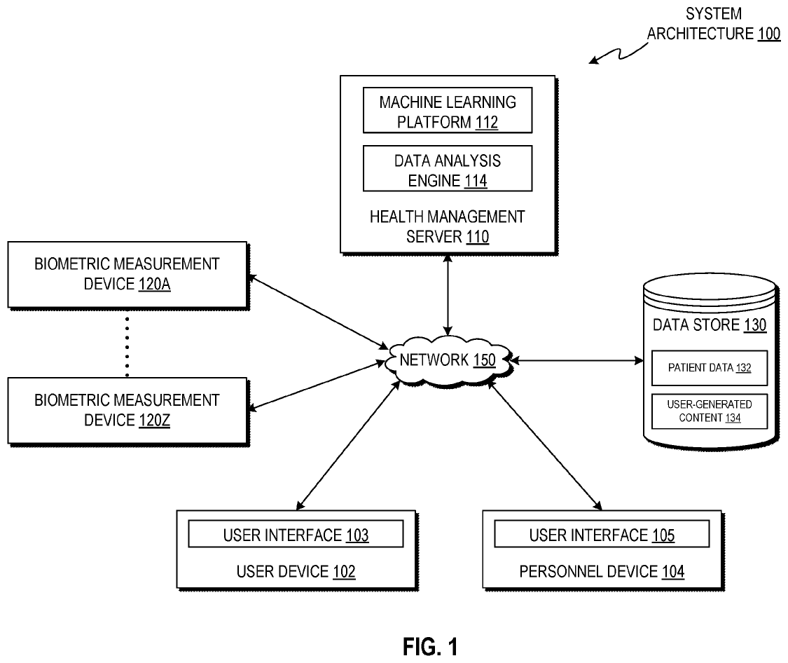

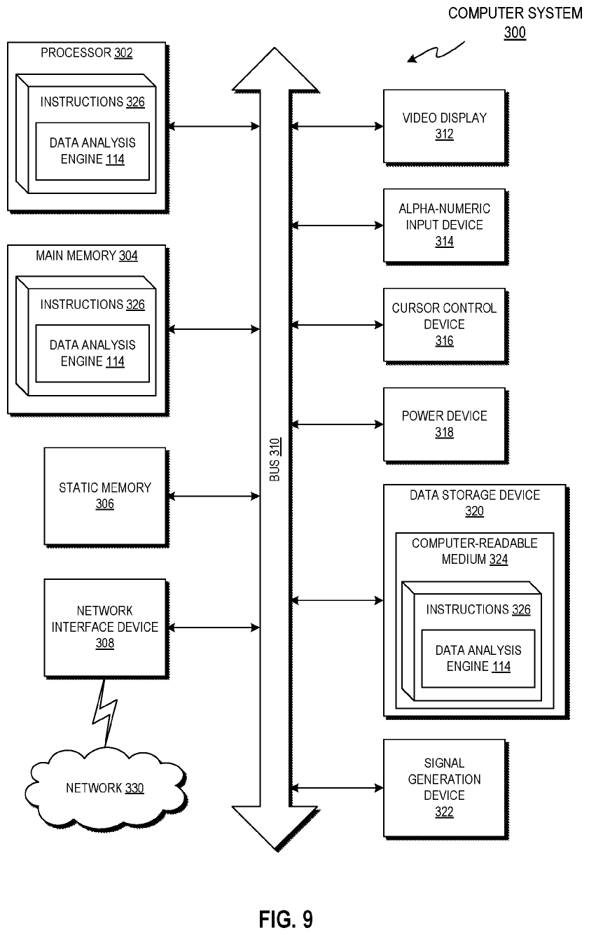

FIG. 1 illustrates an exemplary system architecture 100, in accordance with an embodiment of the present disclosure. The system architecture 100 includes a user device 102, a personnel device 104, a health management server 110, one or more biometric measurement devices 120A-120Z, and a data store 130, with each device of the system architecture 100 being communicatively coupled via a network 150. One or more of the devices of the system architecture 100 may be implemented using computer system 900, described below with respect to FIG. 9. In some embodiments, one or more of the devices of the system architecture 100 may be hosted in a cloud computing environment.

In one embodiment, network 150 may include a public network (e.g., the Internet), a private network (e.g., a local area network (LAN), a wide area network (WAN), or a Bluetooth network), a wired network (e.g., Ethernet network), a wireless network (e.g., an 802.11 network or a Wi-Fi network), a cellular network (e.g., a Long Term Evolution (LTE) network), routers, hubs, switches, server computers, and/or a combination thereof. Although the network 150 is depicted as a single network, the network 150 may include one or more networks operating as a stand-alone network or in cooperation with each other. The network 150 may utilize one or more protocols of one or more devices that are communicatively coupled thereto. The network 150 may translate protocols to/from one or more protocols of the network devices.

The user device 102 and the personnel device 104 may include any computing devices such as personal computers (PCs), laptops, mobile phones, smart phones, tablet computers, netbook computers, etc. The user device 102 and the personnel device 104 may also be referred herein as “client devices” or “mobile devices.” An individual user (e.g., a patient) may be associated with (e.g., own and/or use) the user device 102. Similarly, an individual user (e.g., a clinician or other individual providing health management services to the patient) may be associated with (e.g., own and/or use) the personnel device 104. As used herein, a “user” may be represented as a single individual or a group of individuals. In some embodiments, one or more of the user device 102 or the personnel device 104 may be wearable devices. It is noted that additional user devices and personnel devices may be included in system architecture 100, with a single user device 102 and personnel device 104 being illustrative.

The user device 102 and the personnel device 104 may each implement user interfaces 103 and 105, respectively, which may allow a user of the respective device to send/receive information to/from each other, the health management server 110, the one or more biometric measurement devices 120A-120Z, the data store 130, or any other device via the network 150. For example, one or more of the user interfaces 103 or 105 may be a web browser interface that can access, retrieve, present, and/or navigate content (e.g., web pages such as Hyper Text Markup Language (HTML) pages). As another example, one or more of the user interfaces 103 or 105 may enable data visualization with their respective device. In some embodiments, one or more of the user interfaces 103 or 105 may be a standalone application (e.g., a mobile “app,” etc.), that allows a user of a respective device to send/receive information to/from each other, the health management server 110, the one or more biometric measurement devices 120A-120Z, the data store 130, or any other device via the network 150.

In some embodiments, the user device 102 and the personnel device 104 may each utilize local data stores in lieu of or in combination with the data store 130. Each of the local data stores may be internal or external devices, and may include one or more of a short-term memory (e.g., random access memory), a cache, a drive (e.g., a hard drive), a flash drive, a database system, or another type of component or device capable of storing data. The local data stores may also include multiple storage components (e.g., multiple drives or multiple databases) that may also span multiple computing devices (e.g., multiple server computers). In some embodiments, the local data stores may be used for data back-up or archival purposes.

In various embodiments discussed herein, a “user” of the user device 102 may be a patient who is in a pregnancy-related period. The user device 102 may be representative of one or more devices owned and operated by a single user/patient, or representative of multiple devices owned and operated by a plurality of different users/patients. Each user device 102 may be utilized by a user/patient to generate content (i.e., user-generated content). User-generated content may include any content generated by the patient during a period for which a health evaluation is being performed, including text-based content, survey data (e.g., answers to survey questions, including pre-defined selectable answers and/or open-ended answers that are written by the patient), audio content, image content, video content, or a combination thereof. For example, user-generated content may include written responses to health-based questionnaires, as well the patient's journal data to describe their feelings and/or symptoms. In some embodiments, user-generated content may include or be supplemented by information describing the patient's nutritional intake. In some embodiments, the user-generated content is stored locally on the user device 102, stored in the data store 130 as user-generated content 134, or stored in the health management server 110.

In some embodiments, the user device 102 may be configured to provide various experiences to the patient during a pregnancy-related period, including digital yoga and meditation videos, virtual reality experiences, augmented reality experiences, and meditation experiences incorporating acoustics (e.g., to increase lactation). In some embodiments, the user device 102 may be configured to perform body language recognition, face recognition, or voice recognition, and transmit related data to the health management server 110 for analysis.

A “user” of the personnel device 104 may be a clinician, a team of clinicians, or any individual or group of individuals associated with a health care organization or related organization (e.g., an insurance organization). The personnel device 104 may be representative of multiple personnel devices each used by the same individual or multiple individuals.

In some embodiments, biometric measurement devices 120A-120Z may include one or more devices for measuring biometric data of a user, including a heart rate monitor, a glucose monitor, a respiratory monitor, an electroencephalograph (EEG) device, an electrodermograph (EDG) device, an electromyograph device (EMG), a temperature monitor, an accelerometer, or any other device capable of monitoring a user's biometric data. The data collection may include, but are not limited to, one or more of heart rate data, body temperature data, body composition data (e.g., body mass index, percent body fat, etc.), hemoglobin level data, cholesterol data, sleep data, blood pressure data, respiratory rate data, blood glucose level data, triglyceride data, movement data, electrodermal activity data, or electrocardiogram data, or electroencephalograph data. In some embodiments, one or more of the biometric measurement devices 120A-120Z may be wearable devices. In some embodiments, one or more of the biometric measurement devices 120A-120Z is a biometric contactless sensor such as, for example, a camera (e.g., optical and/or infrared) that captures and records the patient's facial expressions or movements, a microphone for recording the patient's voice, etc. In some embodiments, one or more of the biometric measurement devices 120A-120Z is a medical measurement device, such as a device generally used by a clinician during a medical evaluation or procedure. In some embodiments, one or more of the biometric measurement devices 120A-120Z are connected directly to the user device 120. In some embodiments, one or more of the biometric measurement devices 120A-120Z are “Internet of Things” (IoT) devices that are accessible via the network 150. In some embodiments, the user device 102 may incorporate therein one or more of the biometric measurement devices 120A-120Z. For example, the user device 102 may be an Apple Watch configured to measure a heart rate of the patient.

In some embodiments, the health management server 110 may include one or more computing devices (such as a rackmount server, a router computer, a server computer, a personal computer, a mainframe computer, a laptop computer, a tablet computer, a desktop computer, etc.), data stores (e.g., hard disks, memories, databases), networks, software components, and/or hardware components. The health management server 110 includes a machine learning platform 112 and a data analysis engine 114 used to derive health indicators from user-generated content.

In some embodiments, the machine learning platform 112 may be configured to apply one or more machine learning models, for example, for the purposes of identifying root causes of patient symptoms and generating physical and mental health risk scores for individuals (which may be utilized to generate priority lists of individuals). In some embodiments, the machine learning platform 112 may be configured to apply one or more NLP models (e.g., sentiment analysis models, word segmentation models, or terminology extraction models) to user-generated content and associate health indicators derived from the user-generated content with biometric data. For example, the machine learning platform 112 may utilize supervised or unsupervised models to generate classifications representative of physical or mental health symptoms and/or corresponding root causes based on the health indicators in combination with various biometric data. The machine learning platform 112 may utilize models comprising, e.g., a single level of linear or non-linear operations, such as a support vector machine (SVM), or a deep neural network (i.e., a machine learning model that comprises multiple levels of linear or non-linear operations). For example, a deep neural network may include a neural network with one or more hidden layers. Such machine learning models may be trained, for example, by adjusting weights of a neural network in accordance with a backpropagation learning algorithm.

In some embodiments, each machine learning model may include layers of computational units (“neurons”) to hierarchically process data, and feeds forward the results of one layer to another layer so as to extract a certain feature from the input. When an input vector is presented to the neural network, it may be propagated forward (e.g., a forward pass) through the network, layer by layer (e.g., computational units) until it reaches an output layer. The output of the network can then be compared to a desired output (e.g., a label), using a loss function. The resulting error value is then calculated for each neuron in the output layer. The error values are then propagated from the output back through the network (e.g., a backward pass), until each neuron has an associated error value that reflects its contribution to the original output.

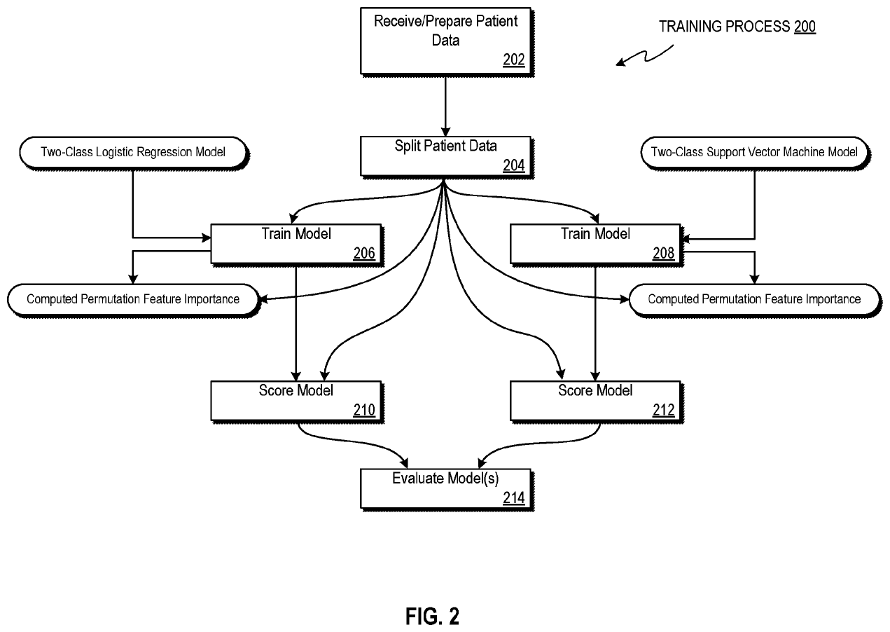

Reference is now made to FIG. 2, which is a flow diagram illustrating a training process 200 for training one or more machine learning models in accordance with at least one embodiment of the present disclosure. In some embodiments, the machine learning platform 112 may utilize a training engine to train the one or more machine learning models. For example, at block 202, the training data may be received and prepared for ingestion by the models. The training data may include a subset of data including, but not limited to, patient biometric data, user-generated content (e.g., standard health survey data), health indicators derived from user-generated content, and EMR data.

At block 204, the patient data is split for the purposes of training multiple models. In some embodiments, the multiple models include a two-class logistic regression model and a two-class support vector machine model, though other models may be utilized, including, but not limited to, random forest models, decision tree models, extreme gradient boosting (XGBoost) models, regularized logistic regression models, multilayer perceptron (MLP) model, naïve Bayes models, and deep learning models. The multiple models are trained at blocks 206 and 208, which may include, but are not limited to, computing permutation feature importance, statistical inference, and principal component analysis. In some embodiments, the training engine may utilize a neural network to train the one or more machine learning models, for example, using a full training set of data multiple times. Each cycle of training is referred to as an “epoch.” For example, each epoch may utilize one forward pass and one backward pass of all training data in the training set. In some embodiments, the machine learning platform 112 may identify patterns in training data that map the training input to the target output (e.g., a particular physical or mental health condition or diagnosis). At blocks 210 and 212, the models are scored, followed by evaluation at block 214.

In some embodiments, a training data generator may also use additional machine learning models to identify and add labels for outcomes based on the training data. The training data generator may utilize a label detector component to detect and generate the labels for the outcomes. The label detector component may also be independent of the training data generator and feed the results to the training data generator. The label detector component may use a machine learning algorithm such as, for example, a neural network, a random decision forest, an SVM, etc., with the training set to detect outcomes. In some embodiments, an NLP model may be used to extract labels from unstructured textual data (e.g., clinician's notes, patient reports, patient history, imaging study reports, etc.). The machine learning platform 112 may learn the patterns from the features, values, and known outcomes and be able to detect similar types of outcomes when provided with comparable set of features and corresponding values. In some embodiments, once the label detector component is sufficiently trained, the label detector may be provided with the features that are made available using the training data generator. The label detector may detect an outcome using the trained machine learning model and produce a label (e.g., “hypertension,” “preeclampsia,” etc.) that is to be stored along with the training data set for the associated machine learning models. In some embodiments, once the outcomes are detected and labels are generated and added for the features and corresponding values, the training data set for the machine learning models may be complete with both inputs and outputs such that the machine learning models may be utilized downstream by the data analysis engine 114.

Referring once again to FIG. 1, in some embodiments, the data analysis engine 114 is configured to detect current physical or mental health conditions and/or predict future physical or mental health conditions based on the outputs of the machine learning platform 112. In some embodiments, the data analysis engine 114 is configured to organize and model data in a manner that allows for visualization during a period over which patient monitoring is performed. In some embodiments, the data analysis engine 114 provides comprehensive physiological data analytics and physiological metrics tracking based on patient biometric data.

In some embodiments, the data analysis engine 114 may generate recommendations for the patient based on the associations made between health indicators and biometric data in order to treat or mitigate symptoms and their underlying causes. For example, such recommendations may include nutritional recommendations, medical procedure or examination recommendations, pharmacological recommendations, complementary or alternative medicine recommendations, exercise recommendations (e.g., breathing exercises), or sleep recommendations (e.g., one or more recommendations/suggestions for improving sleep habits and overall sleep health). Recommendations may be in the form of affirmations, task alerts, clinician matching, or feedback from a health screening tool (e.g., a mood assessment tool). Recommendations may also include product recommendations that are tailored to the particular needs of the patient. In some embodiments, the data analysis engine 114 may track the patient's appointments with clinicians, track the outcomes of such appointments, and provide notifications or reminders of upcoming appointments.

In some embodiments, the data store 130 may include one or more of a short-term memory (e.g., random access memory), a cache, a drive (e.g., a hard drive), a flash drive, a database system, or another type of component or device capable of storing data. The data store 130 may also include multiple storage components (e.g., multiple drives or multiple databases) that may also span multiple computing devices (e.g., multiple server computers). In some embodiments, the data store 130 may be cloud-based. One or more of the devices of system architecture 100 may utilize their own storage and/or the data store 130 to store public and private data, and the data store 130 may be configured to provide secure storage for private data. In some embodiments, the data store 130 may be used for data back-up or archival purposes. In some embodiments, the data store 130 is implemented using a backend Node.js and RESTful API architecture that facilitates rapid and real-time updates of stored data.

In some embodiments, the data store 130 may include patient data 132, which may include biometric data (e.g., measured by one or more of the biometric measurement devices 120A-120Z) or other health-related data of the patient. The data store 130 provides for storage of individual patient data 132 in a HIPAA- and HITRUST-compliant manner, with the patient data 132 including electronic health records (EHRs), physician data, or various other data including surgical reports, imaging data, genomic data, etc. In some embodiments, EHRs are stored in the HL7® FHIR® standard format. In some embodiments, the data store 130 may include the user-generated content 134.

Although each of the devices of the system architecture 100 are depicted in FIG. 1 as single, disparate components, these components may be implemented together in a single device or networked in various combinations of multiple different devices that operate together. In some embodiments, some or all of the functionality of the health management server 110 may be performed by one or more of the user device 102 or the personnel device 104. For example, the user device 102 may implement a software application that performs some or all of the functions of the machine learning platform 112 or the data analysis engine 114.

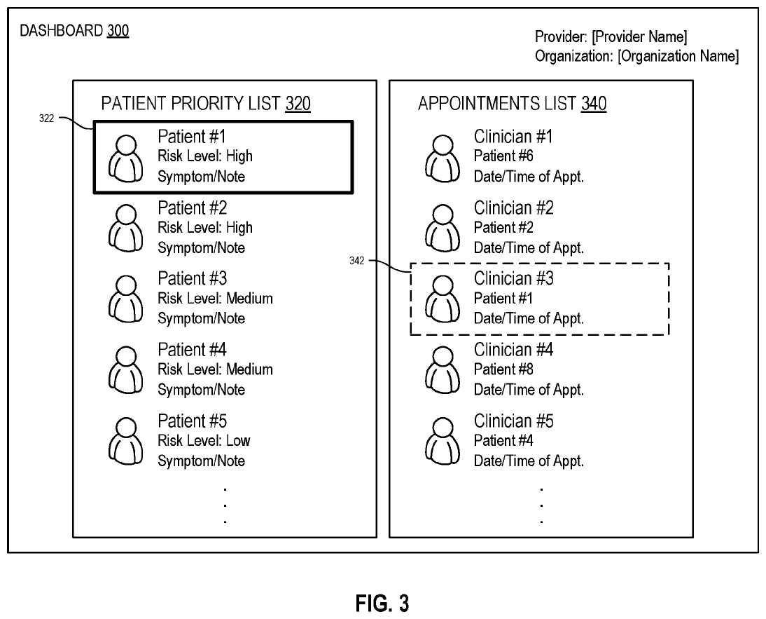

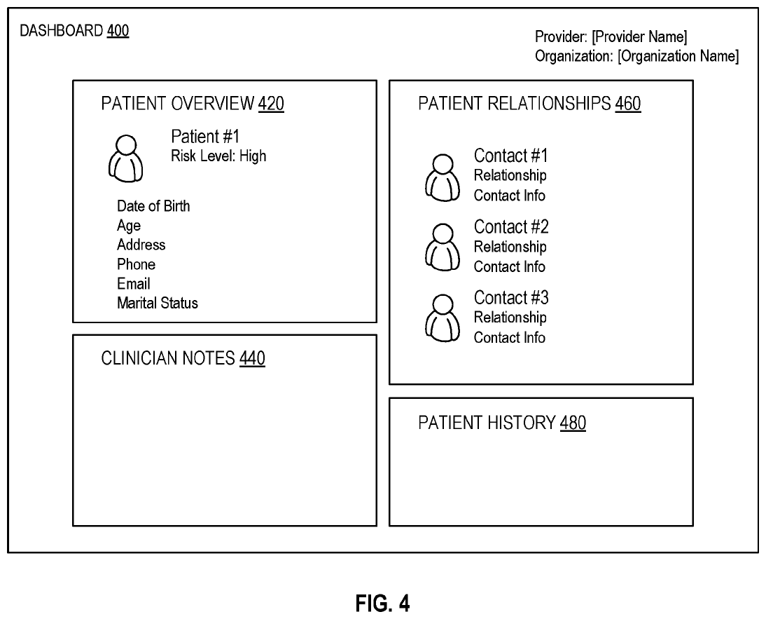

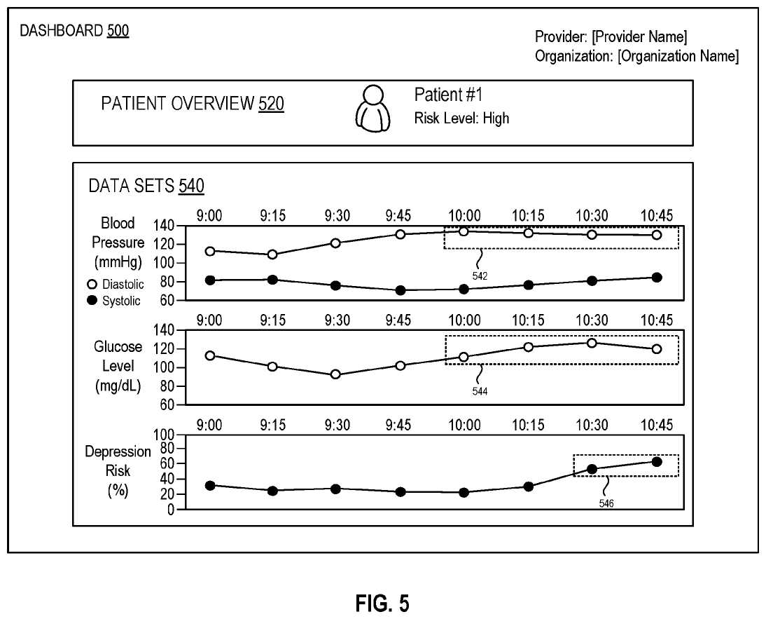

FIGS. 3-5 illustrate exemplary dashboards 300-500 presented for display by the user interfaces 105 of personnel devices 104 in accordance with at least one embodiment of the present disclosure. The dashboards 300-500 are illustrated in the context of a medical practice having multiple clinicians who provide medical services to a plurality of patients. However, it is to be understood that the personnel viewing the dashboards 300-500 are not necessarily clinicians, and the dashboards 300-500 may be tailored to particular personnel such as health plan care managers, or other healthcare stakeholders.

FIG. 3 shows a high level dashboard 300 that lists all patients (or a subset thereof) associated with an organization (e.g., a medical practice) within a patient priority list 320. The patient priority list 320 may be organized to display patients with the highest computed risk scores at the beginning of the list, or displayed in some other suitable manner to draw attention to the high risk patients. Each patient entry in the patient priority list 320 may include a brief summary of the patient, including the patient's name, risk level, one or more notes (e.g., symptoms, an upcoming appointment date, an alert, etc.), and an image of the patient. Within the patient priority list 320, a selected patient 322 is shown, which includes an image 324 of the patient.

The dashboard 300 further includes an appointments list 340 which lists various appointments with each patient. Each entry in the list may be sorted in chronological order, and contain information including the clinician name, the name of the scheduled patient, and the date and time of the appointment. In some embodiments, upon selection of a patient (selected patient 322) in the patient priority list 320, an indicator of the selected appointment 342 associated with that patient may appear in the appointments list 340. In some embodiments, one or more rescheduling operations may be performed automatically and/or at the request of a clinician or other personnel based on patient risk scores/levels. For example, if the selected patient 322 has a risk score that exceeds a threshold risk score, the appointment for this patient may be switched with an earlier patient in the appointments list 340 (e.g., the appointment associated with “Patient #6” due to “Patient #6” having a lower risk than “Patient #1”).

In some embodiments, selection of a patient (i.e., selected patient 322) may result in presentation of the dashboard 400, which provides more detailed information for the selected patient 322. In some embodiments, the dashboard 400 includes a patient overview 420 (which may include the patient name, risk score/level, image, other contact, personal, and/or demographic information), clinician notes 440 entered by the patient's associated clinician, patient relationships 460 (which may include relevant patient contacts, their relationships to the patient, contact info, etc.), and patient history 480 (including past appointments, diagnoses, alerts, etc.).

In some embodiments, additional data pertaining to the selected patient 322 may be presented in the dashboard 500. The dashboard 500 includes a brief patient overview 520 and data sets 540. The data sets 540 may present, for example, time series data related to measured biometric data (e.g., blood pressure, glucose level, or other biometric data as discussed herein), as well as data sets that are derived at least in part from biometric data (e.g., depression risk). The data sets 540 may be captured at regular or irregular intervals (illustrated as 15 minute intervals). In some embodiments, the time points of captured data may not line up, for example, when different biometric measurement devices are operated asynchronously with respect to each other. In some embodiments, alerts 542, 544, and 546 may be displayed to emphasize potentially dangerous deviations (e.g., above a baseline level). In some embodiments, if a clinician is not currently viewing the dashboard 500, a notification may be transmitted to the clinician's device to alert the clinician of the potentially dangerous deviations. In some embodiments, the notification may include a hyperlink directly to the dashboard 500 for the associated patient.

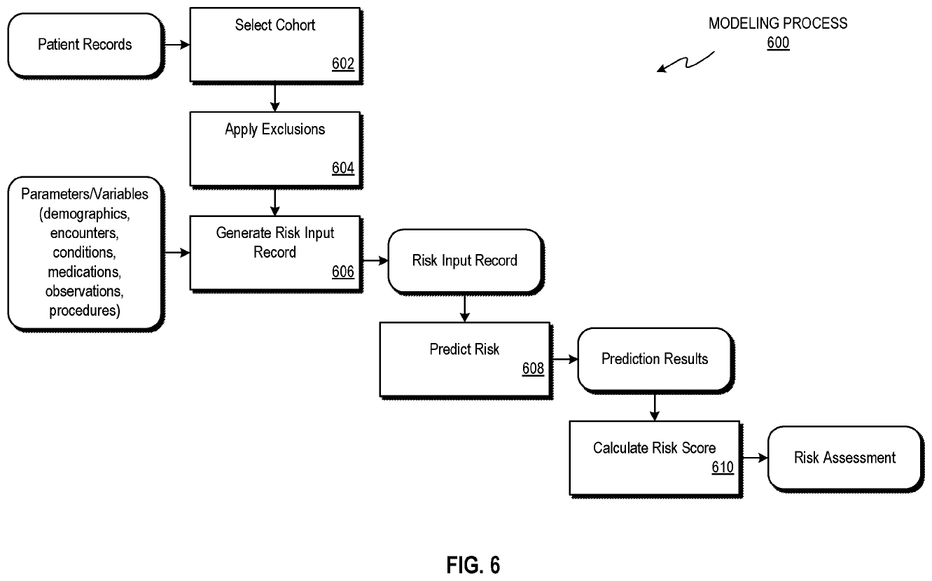

FIG. 6 is a flow diagram illustrating a modeling process 600 for predicting risk and performing risk assessment in accordance with at least one embodiment of the present disclosure. The modeling process 600 may be performed by processing logic that includes hardware (e.g., circuitry, dedicated logic, programmable logic, microcode, etc.), software (e.g., instructions run on a processing device to perform hardware simulation), or a combination thereof. In some embodiments, the modeling process 600 is performed by a processing device of the health management server 110 described with respect to FIG. 1.

Risk score computation may be computed for each new patient cohort, with the result being an overall score for each patient indicating a likelihood of risk of physical health or mental health symptoms or conditions (e.g., a risk of depression). At block 602, a patient cohort is selected, for example, based on one or more selection criteria. The criteria may be that the patients are associated with a particular medical practice, a health plan, an insurance plan, etc. The criteria may be related to demographics (e.g., age, zip code of residence, etc.). Other suitable criteria may be utilized to select the patient cohort. Similarly, once the cohort is chosen, one or more exclusions may be applied at block 604 to remove patients from the cohort.

At block 606, a case record for the cohort is generated by importing parameters and variables associated with each of the patients in the cohort. The parameters/variables may be based on or derived from EMR data, user-generated content, and biometric data. Exemplary variables include, but are not limited to, demographics variables (e.g., age, marital status, etc.), encounters (e.g., emergency visits, etc.), conditions/diagnoses (e.g., abortive outcome, depression, hypertension, migraine, etc.), medications (e.g., antidepressants, etc.), observations (e.g., anxiety, EPDS, SDoH data, sleep data, heart rate variability (HRV) data, etc.), and procedures (e.g., child delivery-related procedures). HRV data, for example, can be useful in developing a prediction model for women at cardiac risk, risk of preeclampsia, and risk of hypertension.

Suitable SDoH variables, which may be derived from EMR data, user-generated content, or other data sources, may include, but are not limited to indicators of economic stability, indicators of education (educational access, quality, highest grade completed, college, post-college education), indicators of health care access (insurance, Medicaid, primary care), indicators of neighborhood and environment (housing, safety, rent), and social and community context indicators (community, family, friends, support, violence).

In certain embodiments, the models described herein recognize and utilize associations between modifiable SDoH variables, race, and sleep which can lead to early actionable clinician recommendations for sleep improvement and subsequently mitigate risk of pregnancy morbidity, particularly for at-risk racial and ethnic groups. Sleep health contributes to physical, mental, and emotional well-being, including gestational hypertension, preeclampsia, gestational diabetes mellitus, mood, attention, and memory. To date, it is unclear how distinct sleep variables such as sleep quantity (fewer than 7 hours or more than 7 hours of sleep per night), sleep quality (the degree to which one has felt refreshed upon waking in the prior 4 months), and sleep-disordered breathing are impacted. Without wishing to be bound by theory, it is believed that specific social determinants of health (SDoH) and race will exhibit different patterns of association with specific sleep variables. This hypothesis impacts maternal comorbidities by specifying specific sleep variables and SDoH variables that can concurrently be targeted in treatment, to increase overall physical and psychological well-being for at-risk racial and ethnic groups. Sleep complications are not generally categorized as maternal morbidities, although they have significant associations with adverse pregnancy outcomes. A treatable condition such as poor sleep is quickly identifiable and can lead to straightforward, actionable steps for a clinician.

Certain embodiments relate sleep duration to several race variables, as a result of a finding that Black and White mothers with shorter sleep duration are at increased risk of morbidities. Decreased sleep duration and decreased sleep quality were associated with discrimination and identifying as Asian. Further, it has been found that Black individuals are more likely to experience deleterious sleep impact both for sleep duration as well as for sleep-disordered breathing. Hispanic individuals are also at increased risk for sleep-disordered breathing. The factors involved may be attributable to variables other than systemic inflammation measured by C-reactive protein. Sleep quality was the sleep variable related most closely to SDoH variables.

In one embodiment, a composite sleep health index may be computed and used as a model input variable. The composite sleep health index may be computed based, for example, on sleep disordered breathing, sleep time, and sleep quality, each of which may be obtained or derived from biometric data and/or user-generated content (e.g., a sleep survey).

At block 608, risks for each patient in the cohort are predicted by utilizing the variables in the risk input record as inputs to one or more of the trained machine learning models described herein. The prediction results are then used to calculate risk scores at block 610, which may be expressed as percentage values in some embodiments. In some embodiments, the risk scores may be normalized based on the risk scores computed for the cohort. In some embodiments, each patient may have one or more associated risk scores that each relate to risk of the patient developing a particular condition (e.g., depression, preeclampsia, or other conditions). Calculated risk scores are then transmitted to one or more personnel devices (e.g., the personnel devices 104).

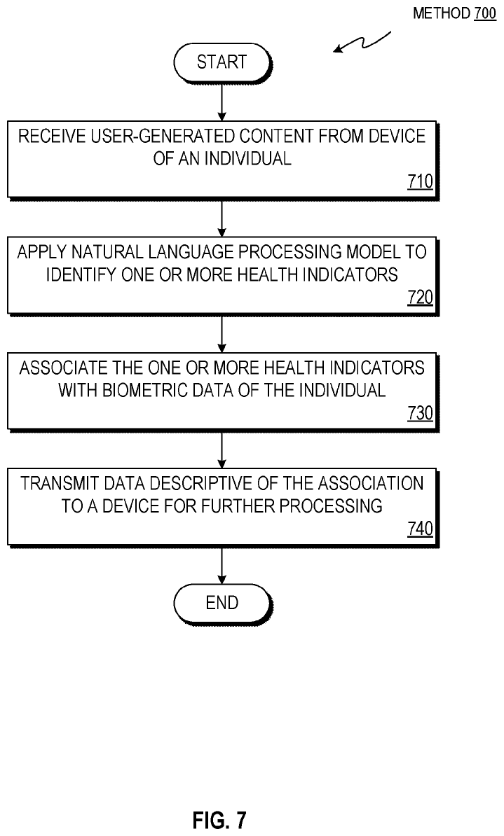

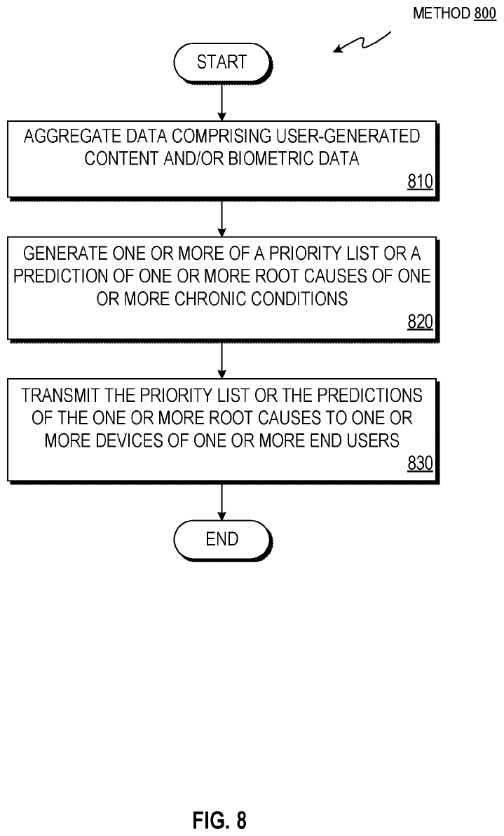

FIGS. 7 and 8 are flow diagrams illustrating exemplary methods 700 and 800, respectively, in accordance with various embodiments of the present disclosure. The methods 700 and/or 800 may be performed by processing logic that includes hardware (e.g., circuitry, dedicated logic, programmable logic, microcode, etc.), software (e.g., instructions run on a processing device to perform hardware simulation), or a combination thereof. In some embodiments, the methods 700 and/or 800 are performed by a processing device of the health management server 110 described with respect to FIG. 1. In other embodiments, some of the functionality of the methods 700 and/or 800 are distributed between the health management server 110 and the user device 102. In other embodiments, the methods 700 and/or 800 are performed by a processing device of the user device 102.

Reference is now made to FIG. 7, which illustrates the method 700 of deriving health indicators from user-generated content in accordance with at least one embodiment. The method 700 begins at block 710, where the processing device receives user-generated content (e.g., user-generated content 134 from the data store 130, from the user device 102, from a separate device such as a content server, or from a combination thereof). In some embodiments, the user-generated content comprises one or more of survey data, digital text (which may include transcribed audio), audio data, video data, or image data. For example, the user-generated content may include journaling data, blog posts, or self-assessments written by the patient (e.g., in the form of digital text, audio data, video data, tablet writing, etc.), medical records, responses to prompt questions (e.g., from a health survey provided to the patient), etc. As another example, the user-generated content may include responses to EPDS, PHQ-9, or GAD-7 questions. In some embodiments, the user-generated content corresponds to content generated by and collected, aggregated, or otherwise received from the patient during a pregnancy-related period of the patient, such as during pregnancy or during a postpartum period.

At block 720, the processing device applies an NLP model (e.g., utilizing the machine learning platform 112) to identify one or more health indicators. In some embodiments, the health indicators include indicators of physical health or mental health symptoms. In some embodiments, the health indicators include indicators of pregnancy-related symptoms, for example, during a pregnancy-related period. In some embodiments, the NLP model utilizes one or more of sentiment analysis, word segmentation, or terminology extraction to identify the one or more health indicators. For example, the NLP model may identify words and phrases that are generally associated with particular symptoms, and/or may evaluate a mental state of the patient based on sentiment of written text in combination with specific words or phrases used by the patient. In some embodiments, a supervised or unsupervised learning model may be used to identify the words and phrases generally associated with particular symptoms via, for example, topic monitoring, clustering, and/or latent semantic indexing.

At block 730, the processing device associates (e.g., utilizing the data analysis engine 114) the one or more health indicators with biometric data of the patient (e.g., biometric data obtained from one or more of the biometric measurement devices 120A-120Z). In some embodiments, the biometric data comprises one or more of heart rate data, body temperature data, body composition data (e.g., body mass index, percent body fat, etc.), hemoglobin level data, cholesterol data, sleep data, blood pressure data, respiratory rate data, blood glucose level data, triglyceride data, movement data, electrodermal activity data, electrocardiogram data, or electroencephalograph data. In some embodiments, the biometric data is received from one or more wearable devices of the patient, one or more biometric contactless sensors, or one or more medical measurement devices.

In some embodiments, the processing device associates the one or more indicators with the biometric data to predict or identify one or more root causes of physical health or mental health symptoms or pregnancy-related symptoms. For example, a woman may indicate that she has been having severe headaches, blurry vision, abdominal pain, or shortness of breath. Key phrase extraction may output the terms “headaches,” “vision,” “breath,” “abdominal, which are all possible indicators of preeclampsia symptoms. However, some symptoms like headaches and pain may generally be overlooked as common pregnancy complaints. In parallel, the biometric data collected could show fluctuations in breathing rate throughout the day or week. For example, if blood pressure has exceeded 140/90 mmHg on two or more occasions at least four hours apart, this a sign of abnormal behavior and a high-risk indicator of preeclampsia. Together, these sets of data observed over a similar time period suggest high risk of, or detection of, preeclampsia in the patient and can prompt early intervention.

In some embodiments, the processing device utilizes a machine learning model (e.g., machine learning platform 112) to associate the one or more indicators with the biometric data. In some embodiments, the machine learning model is trained based on the one or more indicators and the biometric data. In some embodiments, the machine learning model is a supervised machine learning model or an unsupervised machine learning model.

At block 740, the processing device transmits data descriptive of the association to a device for further processing or display to facilitate treating or mitigating one or more root causes of the physical or mental health symptoms, or pregnancy-related symptoms. For example, the processing device (e.g., of the health management server 110) may transmit the data to a clinician's device (e.g., personnel device 104) in a form suitable for visualization and/or further processing. For example, the data may be presented via the user interface 105 of the personnel device 104 in the form of one or more of the dashboards 300, 400, or 500.

In some embodiments, the processing device further generates a recommendation for the patient based at least in part on the association. In some embodiments, the recommendation comprises one or more of a nutritional recommendation, a medical procedure or examination recommendation, a pharmacological recommendation, a complementary or alternative medicine recommendation, an exercise recommendation, or a sleep recommendation.

In some embodiments, the method 700 may iterate through blocks 710, 720, 730, and/or 740 as new user-generated content and biometric data becomes available, for example, at regular intervals or at the request of a clinician, health plan care manager, or other healthcare stakeholder.

Reference is now made to FIG. 8, which illustrates the method 800 of generating priority lists and/or predictions of root causes of acute or chronic conditions in accordance with at least one embodiment. The method 800 begins at block 810, where the processing device aggregating data corresponding to a plurality of individuals (e.g., from user devices 102, biometric measurement devices 120A-120Z, the data store 130, or other data sources). In some embodiments, the data comprises, for each individual, user-generated content and/or biometric data (e.g., as described above with respect to the method 700). In at least one embodiment, the data comprises, for each individual, heart data, sleep data, and user-generated survey data (e.g., a mood log, a symptom log, etc.).

In some embodiments, the aggregation is performed continuously or at regular time intervals (e.g., every 15 minutes, hourly, daily, etc.). In some embodiments, the plurality of individuals correspond to a group of patient's associated with a particular medical practice, healthcare service provider, or healthcare plan. In other embodiments, the plurality of individuals correspond to a group of patients associated with multiple medical practices, healthcare service providers, and/or healthcare plans who are identified based on one or more common attributes or parameters shared by the individuals (e.g., demographics parameters, residence location, physical or mental health conditions or diagnoses, medications, medical procedures, observations, etc.). In some embodiments, the data for each individual corresponds to data generated during a pregnancy-related period of the individual. In some embodiments, the data may be generated during a period related to a treatment, such as treatment for substance abuse, treatment for diabetes, treatment for cancer, etc.

At block 820, the processing device generates, from a machine learning model that utilizes the aggregated user-generated content and/or biometric data as input, one or more of: a priority list for the plurality of individuals; or, for each individual, a prediction, diagnosis, or identification of one or more root causes of one or more acute or chronic conditions of the individual. In some embodiments, the machine learning model is selected from a two-class logistic regression model, a random forest model, a decision tree model, an extreme gradient boosting (XGBoost) model, a regularized logistic regression model, a multilayer perceptron (MLP) model, a support vector machine model, a naïve Bayes model, or a deep learning model.

In some embodiments, the priority list is representative of a health risk for each of the plurality of individuals. The health risk may correspond to risk related to physical health, mental health, or another type of health. An output of the machine learning model may include a risk score (e.g., a numerical score or nomogram), which is used to organize a listing of the individuals in the priority list (e.g., as illustrated by patient priority list 320 of FIG. 3). For example, the risk score may be converted to a category (e.g., “high risk,” “medium risk,” “low risk,” etc.) when presented for display in the priority list.

In some embodiments, risk scores are computed for different time periods associated with a given individual's health conditions. An individual in a pregnancy-related period may have different risk scores associated with different time periods during the pregnancy-related period. For example, data collected during preconception may be used to predict depression in the first trimester of pregnancy. Data obtained during preconception and the first trimester can used to predict depression in the second trimester of pregnancy. Data obtained during preconception, the first trimester, and the second trimester can be used to predict depression in the third trimester. All of the data collected prior to childbirth can then be used to predict postpartum in the fourth trimester and beyond.

In some embodiments, the one or more acute or chronic conditions may correspond to physical or mental health conditions or symptoms. Mental health conditions can include, but are not limited to, depression and other mood disorders. In at least one embodiment, the one or more acute or chronic conditions are predicted, diagnosed, or identified based on HRV data and/or sleep data in combination with the individual's survey data. In some embodiments, the physical or mental health conditions may correspond to those that occurred or are occurring during a pregnancy-related period of the individual, and may relate to chronic or acute pregnancy-related symptoms.

In some embodiments, the user-generated content for at least one individual comprises digital text, which may be text directly entered by the individual on a respective device or transcribed text (“audio-to-text”) from an audio recording of the individual speaking. In such embodiments, the processing device may apply an NLP model to the digital text to identify one or more indicators of physical or mental health conditions or symptoms (e.g., pregnancy-related symptoms during a pregnancy-related period).

In some embodiments, the processing device uses the machine learning model or a different machine learning model to generate, for each individual, SDoH data using the machine learning model or using a different machine learning model that utilizes the one or more of electronic medical records of the individual or the user-generated content as input. In some embodiments, the processing device extracts the SDoH data from electronic medical record (EMR) data and/or user-generated content (e.g., survey data) of the individual.

In some embodiments, for at least one individual, the processing device generates a recommendation for the individual based at least in part on the prediction, diagnosis, or identification of the one or more root causes. In some embodiments, the recommendation comprises one or more of a nutritional recommendation, a medical procedure or examination recommendation, a pharmacological recommendation, a complementary or alternative medicine recommendation, an exercise recommendation, or a sleep recommendation.

At block 830, the processing device transmits the priority list or the prediction(s), diagnosis, or identification(s) of the one or more root causes to one or more devices of one or more end users who are different from the plurality of individuals. In some embodiments, the processing device additionally, or alternatively, transmits data descriptive of the one or more root causes to the one or more devices of the one or more end users for further processing or display to facilitate treating or mitigating the one or more root causes of the physical or mental health symptoms.

For simplicity of explanation, the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term “article of manufacture,” as used herein, is intended to encompass a computer program accessible from any computer-readable device or storage media.

FIG. 9 illustrates a diagrammatic representation of a machine in the exemplary form of a computer system 900 within which a set of instructions (e.g., for causing the machine to perform any one or more of the methodologies discussed herein) may be executed. In alternative embodiments, the machine may be connected (e.g., networked) to other machines in a LAN, an intranet, an extranet, or the Internet. The machine may operate in the capacity of a server or a client machine in client-server network environment, or as a peer machine in a peer-to-peer (or distributed) network environment. The machine may be a personal computer (PC), a tablet PC, a set-top box (STB), a personal digital assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Some or all of the components of the computer system 900 may be utilized by or illustrative of any of the user device 102, the personnel device 104, the health management server 110, the biometric measurement devices 120A-120Z, and the data store 130.

The exemplary computer system 900 includes a processing device (processor) 902, a main memory 904 (e.g., read-only memory (ROM), flash memory, dynamic random access memory (DRAM) such as synchronous DRAM (SDRAM) or Rambus DRAM (RDRAM), etc.), a static memory 906 (e.g., flash memory, static random access memory (SRAM), etc.), and a data storage device 920, which communicate with each other via a bus 910.

In some embodiments, the exemplary computer system 900 may further include a graphics processing unit (GPU) that comprises a specialized electronic circuit for accelerating the creation and analysis of images in a frame buffer for output to a display device. In some embodiments, because of its special design, a GPU may be faster for processing video and images than a CPU of the exemplary computer system 900. Certain embodiments of the present disclosure that implement one or more convolutional neural networks (CNNs) may benefit by increased performance speed by utilizing a GPU to implement the CNN, which may allow for both local implementation (client side) and remote implementation (server-side).

Processor 902 represents one or more general-purpose processing devices such as a microprocessor, central processing unit, or the like. More particularly, the processor 902 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 902 may also be one or more special-purpose processing devices such as an application specific integrated circuit (ASIC), a field programmable gate array (FPGA), a digital signal processor (DSP), network processor, or the like. The processor 902 is configured to execute instructions 926 for performing any of the methodologies and functions discussed herein, such as the functionality of the data analysis engine 114.

The computer system 900 may further include a network interface device 908. The computer system 900 also may include a video display unit 912 (e.g., a liquid crystal display (LCD), a light-emitting diode (LED) display, a cathode ray tube (CRT), etc.), an alphanumeric input device 914 (e.g., a keyboard), a cursor control device 916 (e.g., a mouse), and a signal generation device 922 (e.g., a speaker).

Power device 918 may monitor a power level of a battery used to power the computer system 900 or one or more of its components. The power device 918 may provide one or more interfaces to provide an indication of a power level, a time window remaining prior to shutdown of computer system 900 or one or more of its components, a power consumption rate, an indicator of whether computer system is utilizing an external power source or battery power, and other power related information. In some embodiments, indications related to the power device 918 may be accessible remotely (e.g., accessible to a remote back-up management module via a network connection). In some embodiments, a battery utilized by the power device 918 may be an uninterruptable power supply (UPS) local to or remote from computer system 900. In such embodiments, the power device 918 may provide information about a power level of the UPS.

The data storage device 920 may include a computer-readable storage medium 924 on which is stored one or more sets of instructions 926 (e.g., software) embodying any one or more of the methodologies or functions described herein. The instructions 926 may also reside, completely or at least partially, within the main memory 904 and/or within the processor 902 during execution thereof by the computer system 900, the main memory 904 and the processor 902 also constituting computer-readable storage media. The instructions 926 may further be transmitted or received over a network 930 (e.g., the network 150) via the network interface device 908.

In some embodiments, the instructions 926 include instructions for one or more software components for implementing one or more of the methodologies or functions described herein. While the computer-readable storage medium 924 is shown in an exemplary embodiment to be a single medium, the terms “computer-readable storage medium” or “machine-readable storage medium” should be taken to include a single medium or multiple media (e.g., a centralized or distributed database, and/or associated caches and servers) that store the one or more sets of instructions. The terms “computer-readable storage medium” or “machine-readable storage medium” shall also be taken to include any transitory or non-transitory medium that is capable of storing, encoding, or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The following embodiments summarize various aspects of the present disclosure in order to provide a basic understanding of such aspects. They are intended to neither identify key or critical elements of the disclosure, nor delineate any scope of the other embodiments of the disclosure or any scope of the claims.

Embodiment 1: A method comprising: aggregating data corresponding to a plurality of individuals, the data comprising, for each individual, user-generated content and/or biometric data; generating, from a machine learning model that utilizes the aggregated user-generated content and/or biometric data as input, one or more of: a priority list for the plurality of individuals, the priority list being representative of a health risk for each of the plurality of individuals; or for each individual, a prediction, diagnosis, or identification of one or more root causes of one or more acute or chronic conditions of the individual; and transmitting the priority list or the prediction, diagnosis, or identification of the one or more root causes to one or more devices of one or more end users who are different from the plurality of individuals.

Embodiment 2: The method of Embodiment 1, further comprising: for each individual, predicting, diagnosing, or identifying one or more root causes of physical or mental health symptoms of the individual using the machine learning model or using a different machine learning model that utilizes the individual's user-generated content and biometric data as input; and transmitting the prediction, diagnosis, or identification of the one or more root causes to the one or more devices of the one or more end users for further processing or display to facilitate treating or mitigating the one or more root causes of the physical or mental health symptoms.

Embodiment 3: The method of any of Embodiment 1 or Embodiment 2, wherein the biometric data for each individual comprises one or more of heart rate data, body temperature data, body composition data, hemoglobin level data, cholesterol data, sleep data, blood pressure data, respiratory rate data, blood glucose level data, triglyceride data, movement data, electrodermal activity data, electrocardiogram data, or electroencephalograph data.

Embodiment 4: The method of Embodiment 3, wherein the biometric data for each individual is received from one or more wearable devices, one or more biometric contactless sensors, or one or more medical measurement devices.

Embodiment 5: The method of any Embodiments 1-4, wherein the machine learning model is selected from a two-class logistic regression model, a random forest model, a decision tree model, an extreme gradient boosting (XGBoost) model, a regularized logistic regression model, a multilayer perceptron (MLP) model, a support vector machine model, a naïve Bayes model, or a deep learning model.

Embodiment 6: The method of any Embodiments 1-5, wherein the user-generated content for each individual comprises one or more of survey data, digital text, audio data, video data, or image data.

Embodiment 7: The method of any Embodiments 1-6, wherein the user-generated content for each individual comprises survey data comprising one or more of a mood log or a symptom log.

Embodiment 8: The method of any Embodiments 1-7, wherein the user-generated content for at least one individual comprises digital text, and wherein the method further comprises: applying a natural language processing (NLP) model to the content to identify one or more indicators of pregnancy-related symptoms during a pregnancy-related period of the individual.

Embodiment 9: The method of any Embodiments 1-8, further comprising: for at least one individual, generating a recommendation for the individual based at least in part on the prediction of the one or more root causes, wherein the recommendation comprises one or more of a nutritional recommendation, a medical procedure or examination recommendation, a pharmacological recommendation, a complementary or alternative medicine recommendation, an exercise recommendation, or a sleep recommendation.

Embodiment 10: The method of any Embodiments 1-9, further comprising, for each individual: generating social determinants of health (SDoH) data using the machine learning model or using a different machine learning model that utilizes the one or more of electronic medical records of the individual or the user-generated content as input; or extracting the SDoH data from electronic medical record (EMR) data and/or user-generated content (e.g., survey data) of the individual.

Embodiment 11: A system comprising: a memory; and a processing device coupled to the memory, the processing device being configured to: aggregate data corresponding to a plurality of individuals, the data comprising, for each individual, user-generated content and/or biometric data; generate, from a machine learning model that utilizes the aggregated user-generated content and/or biometric data as input, one or more of: a priority list for the plurality of individuals, the priority list being representative of a health risk for each of the plurality of individuals; or for each individual, a prediction, diagnosis, or identification of one or more root causes of one or more acute or chronic conditions of the individual; and transmit the priority list or the prediction, diagnosis, or identification of the one or more root causes to one or more devices of one or more end users who are different from the plurality of individuals.

Embodiment 12: The system of Embodiment 11, wherein the processing device is further configured to: for each individual, predict, diagnose, or identify one or more root causes of physical or mental health symptoms of the individual using the machine learning model or using a different machine learning model that utilizes the individual's user-generated content and biometric data as input; and transmit the prediction, diagnosis, or identification of the one or more root causes to the one or more devices of the one or more end users for further processing or display to facilitate treating or mitigating the one or more root causes of the physical or mental health symptoms.

Embodiment 13: The system of either Embodiment 10 or Embodiment 11, wherein the biometric data for each individual comprises one or more of heart rate data, body temperature data, body composition data, hemoglobin level data, cholesterol data, sleep data, blood pressure data, respiratory rate data, blood glucose level data, triglyceride data, movement data, electrodermal activity data, electrocardiogram data, or electroencephalograph data.

Embodiment 14: The system of Embodiment 13, wherein the biometric data for each individual is received from one or more wearable devices, one or more biometric contactless sensors, or one or more medical measurement devices.

Embodiment 15: The system of any of Embodiments 11-14, wherein the machine learning model is selected from a two-class logistic regression model, a random forest model, a decision tree model, an extreme gradient boosting (XGBoost) model, a regularized logistic regression model, a multilayer perceptron (MLP) model, a support vector machine model, a naïve Bayes model, or a deep learning model.

Embodiment 16: The system of any of Embodiments 11-15, wherein the user-generated content for each individual comprises one or more of survey data, digital text, audio data, video data, or image data.

Embodiment 17: The system of any of Embodiments 11-16, wherein the user-generated content for each individual comprises survey data comprising one or more of a mood log or a symptom log.

Embodiment 18: The system of any of Embodiments 11-17, wherein the user-generated content for at least one individual comprises digital text, and wherein the processing device is further configured to: applying a natural language processing (NLP) model to the content to identify one or more indicators of pregnancy-related symptoms during a pregnancy-related period of the individual.

Embodiment 19: The system of any of Embodiments 11-18, wherein the processing device is further configured to: for at least one individual, generate a recommendation for the individual based at least in part on the prediction of the one or more root causes, wherein the recommendation comprises one or more of a nutritional recommendation, a medical procedure or examination recommendation, a pharmacological recommendation, a complementary or alternative medicine recommendation, an exercise recommendation, or a sleep recommendation.

Embodiment 20: The system of any of Embodiments 11-19, wherein the processing device is further configured to, for each individual: generate social determinants of health (SDoH) data using the machine learning model or using a different machine learning model that utilizes the one or more of electronic medical records of the individual or the user-generated content as input; or extract the SDoH data from electronic medical record (EMR) data and/or user-generated content (e.g., survey data) of the individual.

Embodiment 21: A non-transitory machine-readable medium having instructions thereon that, when executed by a processing device, cause the processing device to: aggregate data corresponding to a plurality of individuals, the data comprising, for each individual, user-generated content and/or biometric data; generate, from a machine learning model that utilizes the aggregated user-generated content and/or biometric data as input, one or more of: a priority list for the plurality of individuals, the priority list being representative of a health risk for each of the plurality of individuals; or for each individual, a prediction, diagnosis, or identification of one or more root causes of one or more acute or chronic conditions of the individual; and transmit the priority list or the prediction, diagnosis, or identification of the one or more root causes to one or more devices of one or more end users who are different from the plurality of individuals.

Embodiment 22: The non-transitory machine-readable medium of Embodiment 21, wherein the instructions further cause the processing device to: for each individual, predict, diagnose, or identify one or more root causes of physical or mental health symptoms of the individual using the machine learning model or using a different machine learning model that utilizes the individual's user-generated content and biometric data as input; and transmit the prediction, diagnosis, or identification of the one or more root causes to the one or more devices of the one or more end users for further processing or display to facilitate treating or mitigating the one or more root causes of the physical or mental health symptoms.

Embodiment 23: The non-transitory machine-readable medium of either Embodiment 21 or Embodiment 22, wherein the biometric data for each individual comprises one or more of heart rate data, body temperature data, body composition data, hemoglobin level data, cholesterol data, sleep data, blood pressure data, respiratory rate data, blood glucose level data, triglyceride data, movement data, electrodermal activity data, electrocardiogram data, or electroencephalograph data.

Embodiment 24: The non-transitory machine-readable medium of Embodiment 23, wherein the biometric data for each individual is received from one or more wearable devices, one or more biometric contactless sensors, or one or more medical measurement devices.

Embodiment 25: The non-transitory machine-readable medium of any of Embodiments 21-24, wherein the machine learning model is selected from a two-class logistic regression model, a random forest model, a decision tree model, an extreme gradient boosting (XGBoost) model, a regularized logistic regression model, a multilayer perceptron (MLP) model, a support vector machine model, a naïve Bayes model, or a deep learning model.

Embodiment 26: The non-transitory machine-readable medium of any of Embodiments 21-25, wherein the user-generated content for each individual comprises one or more of survey data, digital text, audio data, video data, or image data.

Embodiment 27: The non-transitory machine-readable medium of any of Embodiments 21-26, wherein the user-generated content for each individual comprises survey data comprising one or more of a mood log or a symptom log.

Embodiment 28: The non-transitory machine-readable medium of any of Embodiments 21-27, wherein the user-generated content for at least one individual comprises digital text, and wherein the instructions further cause the processing device to: applying a natural language processing (NLP) model to the content to identify one or more indicators of pregnancy-related symptoms during a pregnancy-related period of the individual.

Embodiment 29: The non-transitory machine-readable medium of any of Embodiments 21-28, wherein the instructions further cause the processing device to: for at least one individual, generate a recommendation for the individual based at least in part on the prediction of the one or more root causes, wherein the recommendation comprises one or more of a nutritional recommendation, a medical procedure or examination recommendation, a pharmacological recommendation, a complementary or alternative medicine recommendation, an exercise recommendation, or a sleep recommendation.

Embodiment 30: The non-transitory machine-readable medium of any of Embodiments 21-29, wherein the instructions further cause the processing device to, for each individual: generate social determinants of health (SDoH) data using the machine learning model or using a different machine learning model that utilizes the one or more of electronic medical records of the individual or the user-generated content as input; or extract the SDoH data from electronic medical record (EMR) data and/or user-generated content (e.g., survey data) of the individual.

Embodiment 31: A method comprising: receiving patient-generated content during a pregnancy-related period of the patient; applying a natural language processing (NLP) model to the content to identify one or more indicators of pregnancy-related symptoms during the pregnancy-related period; and transmitting data descriptive of the indicators to a device for further processing or display to facilitate treating or mitigating one or more root causes of the pregnancy-related symptoms.

Embodiment 32: The method of Embodiment 31, further comprising: associating the one or more indicators with biometric data of the patient measured during the pregnancy-related period to predict or identify the one or more root causes of the pregnancy-related symptoms.

Embodiment 33: The method of Embodiment 32, wherein the biometric data comprises one or more of heart rate data, blood pressure data, blood glucose data, body temperature data, respiratory rate data, body composition data, hemoglobin data, cholesterol data, sleep data, movement data, electrodermal activity data, or electrocardiogram data.

Embodiment 34: The method of Embodiment 32, wherein the biometric data is received from one or more wearable devices of the patient, one or more biometric contactless sensors, or one or more medical measurement devices.

Embodiment 35: The method of either Embodiment 33 or Embodiment 34, wherein associating the one or more indicators with the biometric data of the patient comprises using a machine learning model.

Embodiment 36: The method of any of Embodiments 33-35, further comprising: training a machine learning model based on the one or more indicators and the biometric data.

Embodiment 37: The method of Embodiment 36, wherein the machine learning model is a supervised machine learning model or an unsupervised machine learning model.

Embodiment 38: The method of any of Embodiments 31-37, wherein the NLP model utilizes one or more of sentiment analysis, word segmentation, or terminology extraction.

Embodiment 39: The method of any of Embodiments 31-38, wherein the patient-generated content comprises one or more of digital text, audio data, video data, or image data.

Embodiment 40: The method of any of Embodiments 31-39, further comprising: generating a recommendation for the patient based at least in part on the indicators.

Embodiment 41: The method of Embodiment 40, wherein the recommendation comprises one or more of a nutritional recommendation, a medical procedure or examination recommendation, a pharmacological recommendation, a complementary or alternative medicine recommendation, or an exercise recommendation.

Embodiment 42: A method comprising: receiving patient-generated content; applying a natural language processing model to the patient-generated content to identify one or more indicators of physical health or mental health symptoms; associating the one or more indicators with biometric data of the patient to predict or identify one or more root causes of the physical health or mental health symptoms; and transmitting data descriptive of the association to a device for further processing or display to facilitate treating or mitigating the one or more root causes of the physical or mental health symptoms.

Embodiment 43: The method of Embodiment 42, wherein the NLP model utilizes one or more of sentiment analysis, word segmentation, or terminology extraction.

Embodiment 44: The method of either Embodiment 42 or Embodiment 43, wherein the patient-generated content comprises one or more of digital text, audio data, video data, or image data.

Embodiment 45: The method of any of Embodiments 42-44, wherein the biometric data comprises one or more of heart rate data, blood pressure data, blood glucose data, body temperature data, respiratory rate data, body composition data, hemoglobin data, cholesterol data, sleep data, movement data, electrodermal activity data, or electrocardiogram data.

Embodiment 46: The method of any of Embodiments 42-45, wherein the biometric data is received from one or more wearable devices of the patient, one or more biometric contactless sensors, or one or more medical measurement devices.

Embodiment 47: The method of any of Embodiments 42-46, further comprising: generating a recommendation for the patient based at least in part on the association.

Embodiment 48: The method of Embodiment 47, wherein the recommendation comprises one or more of a nutritional recommendation, a medical procedure or examination recommendation, a pharmacological recommendation, a complementary or alternative medicine recommendation, or an exercise recommendation.

Embodiment 49: The method of any of Embodiments 42-48, wherein associating the one or more indicators with the biometric data of the patient comprises using a machine learning model.

Embodiment 50: The method of any of Embodiments 42-49, further comprising: training a machine learning model based on the one or more indicators and the biometric data.

Embodiment 51: The method of Embodiment 50, wherein the machine learning model is a supervised machine learning model or an unsupervised machine learning model.

Embodiment 52: The method of any of Embodiments 42-51, wherein the physical health or mental health symptoms occur during a pregnancy-related period of the patient.

Embodiment 53: A system comprising: a memory; and a processor, coupled to the memory, the processor to implement the method of any of Embodiments 31-52.

Embodiment 54: A non-transitory machine-readable medium having instructions thereon that, when executed by a processing device, cause the processing device to perform the method of any of Embodiments 31-52.

In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

Some portions of the detailed description may have been presented in terms of algorithms and symbolic representations of operations on data bits within a computer memory. These algorithmic descriptions and representations are the mode through which those skilled in the data processing arts most effectively convey the substance of their work to others skilled in the art. An algorithm is herein, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the preceding discussion, it is appreciated that throughout the description, discussions utilizing terms such as “causing,” “receiving,” “retrieving,” “transmitting,” “computing,” “modulating,” “generating,” “adding,” “subtracting,” “multiplying,” “dividing,” “deriving,” “optimizing,” “calibrating,” “detecting,” “performing,” “analyzing,” “determining,” “enabling,” “identifying,” “diagnosing,” “modifying,” “transforming,” “applying,” “comparing,” “aggregating,” “extracting,” “associating,” “modeling,” “training,” “using,” “implementing,” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

The disclosure also relates to an apparatus, device, or system for performing the operations herein. This apparatus, device, or system may be specially constructed for the required purposes, or it may include a general purpose computer selectively activated or reconfigured by a computer program stored in the computer. Such a computer program may be stored in a computer- or machine-readable storage medium, such as, but not limited to, any type of disk including floppy disks, optical disks, compact disk read-only memories (CD-ROMs), and magnetic-optical disks, read-only memories (ROMs), random access memories (RAMs), EPROMs, EEPROMs, magnetic or optical cards, or any type of media suitable for storing electronic instructions.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application should generally be construed to mean “one or more” unless specified otherwise or clear from context to be directed to a singular form. Reference throughout this specification to “an embodiment,” “one embodiment,” or “some embodiments” means that a particular feature, structure, or characteristic described in connection with the embodiment is included in at least one embodiment. Thus, the appearances of the phrase “an embodiment,” “one embodiment,” or “some embodiments” in various places throughout this specification are not necessarily all referring to the same embodiment. Moreover, it is noted that the “A-Z” notation used in reference to certain elements of the drawings is not intended to be limiting to a particular number of elements. Thus, “A-Z” is to be construed as having one or more of the element present in a particular embodiment.

The present disclosure is not to be limited in scope by the specific embodiments described herein. Indeed, other various embodiments of and modifications to the present disclosure, in addition to those described herein, will be apparent to those of ordinary skill in the art from the preceding description and accompanying drawings. Thus, such other embodiments and modifications are intended to fall within the scope of the present disclosure. Further, although the present disclosure has been described herein in the context of particular embodiments in particular environments for particular purposes, those of ordinary skill in the art will recognize that its usefulness is not limited thereto and that the present disclosure may be beneficially implemented in any number of environments for any number of purposes.

1. A method comprising:
aggregating data corresponding to a plurality of individuals, the data comprising, for each individual, user-generated content and/or biometric data;
generating, from a machine learning model that utilizes the aggregated user-generated content and/or biometric data as input, one or more of:
a priority list for the plurality of individuals, the priority list being representative of a health risk for each of the plurality of individuals; or
for each individual, a prediction, diagnosis, or identification of one or more root causes of one or more acute or chronic conditions of the individual; and
transmitting the priority list or the prediction, diagnosis, or identification of the one or more root causes to one or more devices of one or more end users who are different from the plurality of individuals.

2. The method of claim 1, further comprising:
for each individual, predicting, diagnosing, or identifying one or more root causes of physical or mental health symptoms of the individual using the machine learning model or using a different machine learning model that utilizes the individual's user-generated content and biometric data as input; and
transmitting the prediction, diagnosis, or identification of the one or more root causes to the one or more devices of the one or more end users for further processing or display to facilitate treating or mitigating the one or more root causes of the physical or mental health symptoms.

3. The method of claim 1, wherein the biometric data for each individual comprises one or more of heart rate data, body temperature data, body composition data, hemoglobin level data, cholesterol data, sleep data, blood pressure data, respiratory rate data, blood glucose level data, triglyceride data, movement data, electrodermal activity data, electrocardiogram data, or electroencephalograph data.

4. The method of claim 3, wherein the biometric data for each individual is received from one or more wearable devices, one or more biometric contactless sensors, or one or more medical measurement devices.

5. The method of claim 1, wherein the machine learning model is selected from a two-class logistic regression model, a random forest model, a decision tree model, an extreme gradient boosting (XGBoost) model, a regularized logistic regression model, a multilayer perceptron (MLP) model, a support vector machine model, a naive Bayes model, or a deep learning model.

6. The method of claim 1, wherein the user-generated content for each individual comprises one or more of survey data, digital text, audio data, video data, or image data.

7. The method of claim 1, wherein the user-generated content for each individual comprises survey data comprising one or more of a mood log or a symptom log.

8. The method of claim 1, wherein the user-generated content for at least one individual comprises digital text, and wherein the method further comprises:
applying a natural language processing (NLP) model to the content to identify one or more indicators of pregnancy-related symptoms during a pregnancy-related period of the individual.

9. The method of 1, further comprising:
for at least one individual, generating a recommendation for the individual based at least in part on the prediction of the one or more root causes, wherein the recommendation comprises one or more of a nutritional recommendation, a medical procedure or examination recommendation, a pharmacological recommendation, a complementary or alternative medicine recommendation, an exercise recommendation, or a sleep recommendation.

10. The method of claim 1, further comprising, for each individual:
generating social determinants of health (SDoH) data using the machine learning model or using a different machine learning model that utilizes the one or more of electronic medical records of the individual or the user-generated content as input; or
extracting the SDoH data from electronic medical record (EMR) data and/or user-generated content (e.g., survey data) of the individual.

11-30. (canceled)

31. A method comprising:
receiving patient-generated content during a pregnancy-related period of the patient;
applying a natural language processing (NLP) model to the content to identify one or more indicators of pregnancy-related symptoms during the pregnancy-related period; and
transmitting data descriptive of the indicators to a device for further processing or display to facilitate treating or mitigating one or more root causes of the pregnancy-related symptoms.

32. The method of claim 31, further comprising: associating the one or more indicators with biometric data of the patient measured during the pregnancy-related period to predict or identify the one or more root causes of the pregnancy-related symptoms.

33. The method of claim 32, wherein the biometric data comprises one or more of heart rate data, blood pressure data, blood glucose data, body temperature data, respiratory rate data, body composition data, hemoglobin data, cholesterol data, sleep data, movement data, electrodermal activity data, or electrocardiogram data.

34. The method of claim 32, wherein the biometric data is received from one or more wearable devices of the patient, one or more biometric contactless sensors, or one or more medical measurement devices.

35. The method of claim 1, wherein associating the one or more indicators with the biometric data of the patient comprises using a machine learning model.

36. The method of claim 32, further comprising: training a machine learning model based on the one or more indicators and the biometric data.

37. The method of claim 36, wherein the machine learning model is a supervised machine learning model or an unsupervised machine learning model.

38. The method of claim 31, wherein the NLP model utilizes one or more of sentiment analysis, word segmentation, or terminology extraction.

39-41. (canceled)

42. A method comprising:
receiving patient-generated content;
applying a natural language processing model to the patient-generated content to identify one or more indicators of physical health or mental health symptoms;
associating the one or more indicators with biometric data of the patient to predict or identify one or more root causes of the physical health or mental health symptoms; and
transmitting data descriptive of the association to a device for further processing or display to facilitate treating or mitigating the one or more root causes of the physical or mental health symptoms.

43-54. (canceled)
